(19) United States
(12) Patent Application Publication
(10) Pub. No.: US 2010/0041935 A1
(43) Pub. Date: Feb. 18, 2010

(54) USE OF A CATALYST BASED ON ITQ-6 ZEOLITE TO ISOMERIZE AN AROMATIC C8 CUT

(76) Inventors: Gil Mabilon, Charly (FR); Emmanuelle Guillon, Vernaison (FR); Eric Sanchez, Saint Genis Laval (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/302,115

(22) PCT Filed: May 10, 2007
(86) PCT No.: PCT/FR2007/000796

§ 371 Date: Oct. 28, 2009

(30) Foreign Application Priority Data
May 30, 2006 (FR) 0604927

(51) Int. Cl. C07C 5/22

(52) U.S. Cl. 585/481

(57) ABSTRACT

A process for isomerizing an aromatic C8 cut uses a catalyst which comprises:
at least one ITQ-6 zeolite;
at least one metal from group VIII of the periodic table;
at least one binder;
optionally, at least one metal selected from the group formed by elements from groups IIIA, IVA and VIIB of the periodic table; and
optionally, sulphur.

The present invention relates to the isomerization of mixtures of aromatic compounds containing 8 carbon atoms, hereinafter termed “aromatic C8 cuts”.

The aromatic C8 cuts under consideration may be defined as being selected from:
cuts which comprise at least one xylene;
cuts which comprise ethylbenzene; and
cuts which comprise mixtures of at least one xylene and ethylbenzene.

More particularly, the present invention relates to a process for isomerizing an aromatic C8 cut aimed at maximizing the production of para-xylene.

Catalysis of the isomerization of ethylbenzene into xylenes necessitates the presence of a metal from group VIII. Optimized formulations based on mordenite and a metal from group VIII result in catalysts with which side reactions remain non negligible. An example which may be cited is naphthene ring opening, which may or may not be followed by cracking, or disproportionation and transalkylation reactions of C8 aromatics, which lead to the formation of unwanted aromatics. Thus, it would be particularly advantageous to discover more selective, novel catalysts.

Zeolites used in isomerizing aromatic C8 cuts comprise ZSM-5, used alone or as a mixture with other zeolites such as mordenite, for example. Such catalysts have been described in particular in United States and European patents U.S. Pat. No. 4,467,129, U.S. Pat. No. 4,482,773 and EP-A-0 013 617. Other catalysts, principally based on mordenite, have been described, for example, in patent documents U.S. Pat. No. 4,723,051, U.S. Pat. No. 4,665,258 and FR-A-2 477 903. More recently, a catalyst based on a zeolite with structure type EUO has been proposed (EP-A1-0 923 987).

The lack of selectivity of mordenite may be attenuated by optimizing formulations and/or by specific treatments, such as that described in the Applicant's patent FR-B-2 691 914, for example. Those techniques can reduce unwanted disproportionation reactions.

International patent application WO-A-2005/065380 describes the use of a zeolite with structure type MTW for the isomerization of xylenes and ethylbenzene.

The Applicant has now surprisingly discovered a catalyst, preferably in the form of extrudates or beads, used in aromatic C8 cut isomerization reactions, containing at least one ITQ-6 zeolite, a binder and at least one metal from group VIII of the periodic table (advantageously well dispersed on the surface of the catalyst and macroscopically well dispersed through the catalyst grain) which can produce xylene isomerization performances which are remarkable in terms of activity, selectivity and also in terms of stability over time.

Hence, the invention provides an improved process for isomerizing xylenes in an aromatic C8 cut in the presence of a catalyst which comprises:
at least one ITQ-6 zeolite;
at least one metal from group VIII of the periodic table (Handbook of Physics and Chemistry, 76^{th} edition);
at least one binder;
optionally, at least one metal selected from the group formed by elements from groups IIIA, IVA and VIIB of the periodic table; and
optionally, sulphur.

More particularly, the catalyst used in the aromatic C8 cut isomerization process of the present invention is in the form of beads or extrudates.

The structure of ITQ-6 zeolite has not yet been defined. It is a delaminated zeolite derived from the lamellar preferrierite. The synthesis of ITQ-6 zeolite and its physico-chemical characteristics have in particular been described in EP-B1-1 102 630. Further, Corma et al, who discovered that solid; have shown in an article in Angewandte Chemie Int Ed, 39 (2000), 8, p 1499, that ITQ-6 is stable and has a large external surface area. Further, ITQ-6 zeolite has advantageous catalytic properties for hydrocarbon cracking reactions, in particular of sterically hindered molecules. EP-B1-1 102 630 describes the use of ITQ-6 zeolite in isodewaxing processes, in the isomerization of alkenes and in cracking hydrocarbons at high temperature and pressure.

The zeolite forming part of the composition of the catalyst used in the invention is an ITQ-6 zeolite which comprises silicon and at least one element T selected from the group formed by aluminium, iron, gallium and boron, preferably aluminium and boron, with an atomic ratio Si/T of 2:1 to 100:1, preferably 5:1 to 80:1 and more preferably 5:1 to 50:1.

In the catalyst used in the invention, the ITQ-6 zeolite is at least partially, preferably practically entirely in the acid form, i.e. in the hydrogen form (H⁺), the sodium content preferably being such that the atomic ratio Na/T is less than 0.5:1, preferably less than 0.1:1, and more particularly less than 0.02:1.

The ITQ-6 zeolite forming part of the composition of the catalyst used in the invention generally has a total surface area, measured by nitrogen chemisorption using the BET method, of more than 300 m²/g. Preferably, its total surface area is more than 400 m²/g.

The binder (or matrix) forming part of the composition of the catalyst used in the invention generally consists of at least one element selected from the group formed by clays, magnesia, aluminas, silicas, titanium oxide, boron oxide, zirconia, aluminium phosphates, titanium phosphates, zirconium phosphates and silica-aluminas. Coal may also be used. Preferably, the binder is alumina.

The group VIII metal present in the catalyst used in accordance with the invention may be any metal selected from iron, cobalt, nickel, ruthenium, rhodium, palladium, osmium, iridium and platinum. It preferably consists of a noble metal such as palladium or platinum. More preferably, it is platinum.

Depending on the method employed for deposition, as will be indicated below, the noble group VIII metal may be deposited mainly on the zeolite or on the binder.

Advantageously, said metal will be well dispersed on the surface of the catalyst and macroscopically well dispersed through the grain of the catalyst.

The catalyst used in accordance with the invention may also optionally contain at least one metal selected from the group formed by elements from groups IIIA IVA and VIIB of the periodic table and selected, for example, from gallium, indium, tin and rhenium. Preferably, this metal is indium, tin or rhenium.

In the composition of the catalyst used in the invention, the ITQ-6 zeolite is more particularly in an amount of 1% to 90%, preferably 3% to 80%, more preferably 4% to 60% by weight with respect to the weight of catalyst. The group VIII metal, deposited on the zeolite or on the binder, represents 0.01% to 4%, preferably 0.05% to 2.0% by weight with respect to the catalyst weight. The binder constitutes the complement to 100%.

When the catalyst used in the invention contains at least one metal selected from metals from groups IIIA, IVA and VIIB, the amount thereof may be up to 2% by weight with respect to the catalyst weight. It is thus advantageously 0.01% to 2%, preferably 0.05% to 1.0% by weight.

When the catalyst used in the invention contains sulphur, the amount thereof may be such that the ratio of the number of sulphur atoms to the number of atoms of group VIII metal deposited is up to 2:1. It is advantageously 0.5:1 to 2:1.

The catalyst used in the invention may be prepared using any method which is known to the skilled person.

The group VIII metal may be introduced onto the zeolite or the binder before or after forming. When a plurality of metals is introduced, these may be introduced either all in the same manner or using different techniques, at any moment in the preparation, before or after forming and in any order.

However, the preferred method for preparing the catalyst used in the invention consists of forming the mixture of zeolite and binder before depositing the group VIII metal and optional metal from groups IIIA, IVA or VIIB.

More particularly, forming consists of mixing the ITQ-6 zeolite in a wet gel of matrix (generally obtained by mixing at least one acid and a powdered matrix), for example alumina, for the period required to obtain good homogeneity for the paste, i.e. for about ten minutes, then passing the paste obtained through a die to form extrudates, for example with a diameter of 0.4 to 4 mm.

Forming is generally followed by drying (for example for a few hours at about 120° C. in an oven) and calcining, generally at a temperature of 250° C. to 600° C. (for example two hours at about 400° C.).

After calcining, at least one element from group VIII is preferably introduced by selective deposition onto the zeolite-binder mixture using any process which is known to the skilled person. As an example, deposition is carried out using the dry impregnation technique, the excess impregnation technique or, more particularly, by ion exchange.

Deposition is advantageously carried out so that the dispersion of said element(s), determined by chemisorption, is 50% to 100%, preferably 60% to 100% and more preferably 70% to 100%.

It is also preferred to obtain good distribution of said element(s) in the formed catalyst. Said distribution is characterized by its profile obtained by a Casting microprobe. The ratio of the concentrations of each group VIII element in the core of the grain with respect to the edge of that grain, defined as the distribution coefficient, is advantageously from 0.7:1 to 1.3:1, preferably from 0.8:1 to 1.2:1.

Any precursor is suitable for depositing said elements. As an indication, platinum may be introduced in the form of hexachloroplatinic acid, but for any noble group VIII metal, it is also possible to use ammoniacal compounds or compounds such as ammonium chloroplatinate, dicarbonyl platinum dichloride, hexahydroxyplatinic acid, palladium chloride or palladium nitrate.

As will be indicated below, the choice of precursor and the mode of deposition is made as a function of the desired localization of the metal in (or on) the zeolite and/or in (or on) the binder.

Controlling certain parameters which come into play during deposition, in particular the nature of the precursor of the group VIII element(s) used, allows said element(s) to be deposited mainly on the binder or on the zeolite.

Thus, to introduce the group VIII metal, for example platinum or palladium, anionic exchange may be carried out with hexachloroplatinic acid and/or hexachloropalladic acid, in the presence of a competing agent, for example hydrochloric acid, deposition generally being followed by calcining, for example for about 2 hours at about 400° C.

With such precursors, the metal is mainly deposited on the binder and it has good dispersion and good macroscopic distribution through the catalyst grain.

It is also possible to envisage depositing the group VIII metal by cationic exchange. In the case of platinum, then, the precursor may, for example, be selected from:
ammoniacal compounds such as tetrammine platinum (II) salts with formula Pt(NH₃)₄X₂, hexammine platinum (IV) salts with formula Pt(NH₃)₆X₄; halogenopentammine platinum (IV) salts with formula Pt(NH₃)₅X₃; N-tetrahalogenodiammine platinum salts with formula PtX₄ (NH₃)₂; and
halogenated compounds with formula (H(Pt(acac)₂X);
   where X is a halogen selected from the group formed by chlorine, fluorine, bromine and iodine, X preferably being chlorine, and “acac” representing an acetylacetonate group (with empirical formula C₅H₇O₂), a derivative of acetylacetone.

In this case, the group VIII metal is deposited mainly on the zeolite. It has good dispersion and good macroscopic distribution through the catalyst grain.

When a plurality of group VIII elements are to be deposited on the catalyst, said metals may be introduced either all in the same manner or using different techniques and in any order.

The noble metal from the platinum family is preferably introduced by impregnation using an aqueous or organic solution of one of the precursors cited above.

Examples of organic solvents which may be cited are paraffinic, naphthenic or aromatic hydrocarbons containing 6 to 12 carbon atoms per molecule, for example, and organic halogenated compounds containing 1 to 12 carbon atoms per molecule, for example. Examples which may be cited are n-heptane, methylcyclohexane, toluene and chloroform. It is also possible to use mixtures of solvents.

In the catalyst used in the invention, when at least one metal from groups IIIA, IVA and VIIB is to be introduced, any deposition technique which is known to the skilled person and any precursor may be suitable.

It is possible to add the group VIII elements and those from groups IIA, IVA and VIIB, either separately at any stage of the preparation of said catalyst, or simultaneously in at least one unitary step. When at least one element from groups IIIA, IVA and VIIB is added separately, it is preferable for it to be added after the group VIII element.

The additional metal may be introduced by dint of compounds such as chlorides, bromides and nitrates of elements from groups IIIA, IVA and VIIB. As an example, in the case of indium, the nitrate or chloride is advantageously used, and in the case of rhenium, perrhenic acid is advantageously used.

This metal may also be introduced in the form of at least one organic compound selected from the group constituted by complexes of said metal, in particular polyketone complexes of the metal and hydrocarbyl metals such as metal alkyls, cycloalkyls, aryls, alkylaryls and arylalkyls. In this latter case, the metal is advantageously introduced using a solution of an organometallic compound of said metal in an organic solvent. It is also possible to use organohalogenated compounds of the metal. Organic metal compounds which may in particular be cited are tetrabutyltin in the case of tin and triphenylindium in the case of indium.

If the additional metal is introduced before the group VIII metal, the metal compound used is generally selected from the group constituted by the metal halide, nitrate, acetate, tartrate, carbonate and oxalate. Introduction is advantageously carried out in aqueous solution. However, it may also be introduced using a solution of an organometallic compound of the metal, for example tetrabutyltin. In this case, before proceeding to introduce at least one group VIII metal, calcining is carried out in air.

Further, intermediate treatments such as calcining and/or reduction may be applied between successive deposits of the various metals.

Catalyst preparation is generally completed by calcining, normally at a temperature of about 250° C. to 600° C., for a period of about 0.5 to 10 hours, preferably preceded by drying, for example oven drying, at a temperature from ambient temperature to 250° C., preferably 40° C. to 200° C. Said drying step is preferably carried out during the temperature ramp-up necessary to carry out said calcining.

It is possible to carry out prior in situ reduction of the catalyst in a stream of hydrogen, for example at a temperature of 450° C. to 600° C., for a period of 0.5 to 4 hours.

In the case in which the catalyst contains no sulphur, the metal is reduced in hydrogen in situ before injecting the feed.

In the case in which the catalyst used in the invention contains sulphur, the sulphur is preferably introduced onto the catalyst which has been formed, calcined, containing the metals cited above, either in situ before the catalytic reaction or ex situ. Optional sulphurization is carried out after reduction. In the case of in situ sulphurization, if the catalyst has not been reduced, reduction is carried out before sulphurization. In the case of ex situ sulphurization, reduction then sulphurization is carried out. Sulphurization is carried out in the presence of hydrogen using any well known sulphurization agent, such as dimethyldisulphide or hydrogen sulphide. As an example, the catalyst is treated with a feed containing dimethyldisulphide in the presence of hydrogen, with a concentration such that the sulphur/metal atomic ratio is 1.5. The catalyst is then kept for about 3 hours at about 400° C. in a flow of hydrogen before injecting the feed.

The catalyst used in the invention may be employed in reactions for isomerizing an aromatic C8 cut comprising, for example, either a mixture of xylenes alone or ethylbenzene alone or a mixture of xylene(s) and ethylbenzene. Said process is generally carried out under the following operating conditions:
a temperature of 300° C. to 50° C., preferably 320° C. to 450° C. and more preferably 340° C. to 430° C.;
a partial pressure of hydrogen of 0.3 to 1.5 MPa, preferably 0.4 to 1.2 MPa and more preferably 0.7 to 1.2 MPa;
a total pressure of 0.45 to 1.9 MPa, preferably 0.6 to 1.5 MPa; and
an hourly space velocity, expressed in kilograms of feed introduced per kilogram of catalyst per hour, of 0.25 to 30 h⁻¹, preferably 1 to 10 h⁻¹ and more preferably 2 to 6 h⁻¹.

The catalyst used in the invention, formed into strong beads or extrudates, constituted by at least one ITQ-6 zeolite, at least one binder, at least one metal selected from elements from group VIII of the periodic table, said metal being well dispersed on the surface of the catalyst and macroscopically well distributed across the grain of the catalyst, has excellent catalytic performance as regards hydrocarbon transformations, in terms of activity, selectivity and stability over time, such as the isomerization of aromatic C8 cuts, i.e. mixtures constituted by xylenes and, optionally, ethylbenzene.

The following examples illustrate the invention without in any way limiting its scope.

### EXAMPLE 1

### Preparation of Catalyst Containing ITQ-6 Zeolite and 0.3% by Weight of Platinum

The starting zeolite was an ITQ-6 with a Si/Al ratio of 15, in the H⁺ form. This zeolite was then formed by extrusion (extrusion diameter 1.4 mm) with an alumina gel to obtain, after drying and calcining in dry air, a support S1 which contained 20% by weight of ITQ-6 zeolite in the hydrogen form and 80% by weight of alumina.

Said support S1 underwent anionic exchange with hexachloroplatinic acid in the presence of a competing agent (hydrochloric acid) to deposit 0.3% by weight of platinum with respect to the catalyst. The moist solid was dried at 120° C. for 12 hours and calcined in a flow of dry air at a temperature of 500° C. for one hour.

Catalyst A obtained contained, by weight, 20% of ITQ-6 in the hydrogen form, 79.7% of alumina and 0.3% of platinum.

### EXAMPLE 2

### Evaluation of Catalytic Properties of Catalyst A in Isomerizing an Aromatic C8 Cut

The aromatic C8 cut to be isomerized principally contained meta-xylene, ortho-xylene and ethylbenzene.

The operating conditions for isomerization were as follows:
temperature: 390° C.;
total pressure: 15 bars (1 bar 0.1 MPa);
partial pressure of hydrogen: 12 bars.

Catalyst A obtained as described in Example 1 was reduced at 450° C. for 2 hours in a stream of hydrogen.

The catalyst was pre-treated with a feed containing dimethyldisulphide (DMDS) in the presence of hydrogen, with a concentration such that the sulphur/metal atomic ratio was 1.5. The catalyst was then kept for 3 hours at 400° C. in a stream of hydrogen, then the feed was injected.

The catalyst was evaluated as regards activity (by the approaches to equilibrium of para-xylene and ethylbenzene and by ethylbenzene conversion) and as regards selectivity (by the net losses of para-xylene at iso-approach to equilibrium).

Further, side reactions result in three types of losses: losses to paraffins essentially resulting from naphthene ring opening reactions followed by cracking, losses to aromatics formed by disproportionation and transalkylation reactions of aromatics containing 8 carbon atoms (AC8) and finally, losses to napthenes including naphthenes containing 8 carbon atoms (N8) due to hydrogenation of aromatics. Since the N8s can be recycled, we compare the losses by cracking and disproportionation/transalkylation including napthenes other than N8 (the sum of which constitutes the net losses) taking a base 100 for each of these losses for the catalyst A which is not in accordance with the invention.

To calculate the approaches to equilibrium (AEQ), the concentrations of ethylbenzene (% EB) are expressed with respect to the four AC8 isomers, and those of para-xylene (% pX) are expressed with respect to the three xylene isomers.

The approaches to equilibrium (AEQ) are defined as follows:

pX AEQ(%)=100×(% pX_{effluent}−% pX_{feed})/(% PX_{equilibrium}−/PX_{feed})

EB AEQ(%)=100×(%EB_{effluent}−% EB_{feed})/(% EB_{equilibrium}−% EB_{feed})

The cracking losses (P1, weight %) are the AC8 losses in the form of C1 to C8 paraffins (PAR):

$$\frac{100 \times [ \begin{matrix}( \% {PAR}_{effluent} \times weight of effluent ) - \\ ( \% {PAR}_{feed} \times weight of feed )\end{matrix} ]}{( \% AC {8}_{feed} \times weight of feed )}$$

The disproportionation/transalkylation losses (P2, % by weight) are the AC8 losses in the form of naphthenes other than N8, toluene, benzene and C9+ aromatics (OAN):

$$\frac{100 \times [ \begin{matrix}( \% {OAN}_{effluent} \times weight of effluent ) - \\ ( \% {OAN}_{feed} \times weight of feed )\end{matrix} ]}{( \% A C {8}_{feed} \times weight of feed )}$$

The sum of losses P1 and P2 represents the net losses.

The following results were obtained:

| | |
|---|---|
| pX AEQ (%): | 98.2 |
| EB AEQ (%): | 91.3 |
| EB conversion (%): | 56.4 |
| Net losses (weight %): | 5.4 |

These results show that catalyst A, in accordance with the invention, performs well in isomerizing an aromatic C8 cut.

1. In a process comprising isomerizing an aromatic C8 cut in the presence of a catalyst, the improvement wherein said catalyst comprises:
at least one ITQ-6 zeolite;
at least one metal from group VIII of the periodic table;
at least one binder;
optionally, at least one metal from elements of groups IIIA, IVA or VIIB of the periodic table; and
optionally, sulphur.

2. A process according to claim 1, wherein the ITQ-6 zeolite comprises silicon and at least one element T from the group of aluminium, iron, gallium and boron, wherein the Si/T atomic ratio is 2:1 to 100:1, said zeolite being at least partially acidified.

3. A process according to claim 2, wherein the ITQ-6 zeolite has a sodium content corresponding to a Na/T atomic ratio of less than 0.5:1.

4. A process according to claim 1, wherein the ITQ-6 zeolite has a total surface area, measured by nitrogen chemisorption using the BET method, of more than 300 m²/g.

5. A process according to claim 1, wherein the group VIII metal is platinum or palladium.

6. A process according to claim 5, in which the group VIII metal is platinum.

7. A process according to claim 1, wherein the metal from groups IIA, IVA or VIIB is gallium, indium, tin or rhenium.

8. A process according to claim 1 wherein:
the ITQ-6 zeolite represents 1% to 90% by weight of the catalyst;
the group VIII metal represents 0.01% to 4% by weight of catalyst;
the complement to 100% by weight of the catalyst comprises at least one binder.

9. A process according to claim 1, wherein the group VIII metal exhibits a distribution coefficient, determined for a grain of catalyst, corresponding to a ratio of the concentration of the group VIII metal in the core of the grain with respect to the edge of that grain of 0.7:1 to 1.3:1.

10. A process according to claim 1, wherein the dispersion of the group VIII metal exhibits a dispersion on the zeolite-binder mixture, determined by chemisorption, of 50% to 100%.

11. A process according to claim 1 wherein the group VIII metal is principally deposited on the binder.

12. A process according to claim 1 wherein the group VIII metal is principally deposited on the zeolite.

13. A process according to claim 1, wherein the metal from groups IIIA, IVA or VIIB is present in an amount of up to 2% by weight with respect to the weight of catalyst.

14. A process according to claim 1, wherein said catalyst further comprises sulphur in an amount corresponding to a ratio of the number of sulphur atoms to the number of atoms of group VIII metal of up to 2:1.

15. A process according to claim 1, wherein the isomerizing is carried out under the following operating conditions:
a temperature of 300° C. to 500° C.;
a partial pressure of hydrogen of 0.3 to 1.5 MPa;
a total pressure of 0.45 to 1.9 MPa; and
an hourly space velocity, expressed in kilograms of feed introduced per kilogram of catalyst per hour, of 0.25 to 30 h⁻¹.

16. A process according to claim 1, wherein said aromatic C8 cut comprises:
cuts which comprise at least one xylene;
cuts which comprise ethylbenzene; or
cuts which comprise mixtures of at least one xylene and ethylbenzene.

17. A process according to claim 6, wherein the metal from groups IIIA, IVA or VIIB is gallium, indium, tin or rhenium.

18. A process according to claim 17, wherein the group VIII metal exhibits a distribution coefficient, determined for a grain of catalyst, corresponding to a ratio of the concentration of the group VIII metal in the core of the grain with respect to the edge of that grain of 0.7:1 to 1.3:1.

19. A process according to claim 9, wherein the dispersion of the group VIII metal exhibits a dispersion on the zeolite-binder mixture, determined by chemisorption, of 50% to 100%.

20. A process according to claim 18, wherein the dispersion of the group VIII metal exhibits a dispersion on the zeolite-binder mixture, determined by chemisorption, of 50% to 100%.
